(19) United States
(12) Patent Application Publication
(10) Pub. No.: US 2007/0166270 A1
(43) Pub. Date: Jul. 19, 2007

(54) Emulsifier concentrate for a cosmetic composition

(76) Inventors: Michael Neuss, Koln (DE); Thomas Albers, Dusseldorf (DE)

Correspondence Address: COGNIS CORPORATION;PATENT DEPARTMENT, 300 BROOKSIDE AVENUE, AMBLER, PA, 19002, US

(21) Appl. No.: 11/579,170

(22) PCT Filed: Apr. 20, 2005
(86) PCT No.: PCT/EP05/04197

§ 371 Date: Oct. 30, 2006

(30) Foreign Application Priority Data
Apr. 29, 2004 (DE) 10 2004 021 312.7

(51) Int. Cl. A61K 8/37

(52) U.S. Cl. 424/70.31

(57) ABSTRACT

The invention relates to emulsifier concentrates which are pourable at 20° C. and contain (a) up to 90 percent by weight of a polyethylene glycol fatty acid diester based on a C16-C22 fatty acid, C16-C22 hydroxy fatty acid, polyhydroxy stearic acid, or poly-(C16-C22 hydroxy fatty acid), (b) up to 80 percent by weight of an oil or oil mixture that is liquid at 25° C., and (c) 2 to 8 percent by weight of water. Also disclosed are the use in emulsions and a method for producing emulsions based on the inventive emulsifier concentrates.

### FIELD OF THE INVENTION

This invention relates to emulsifier concentrates pumpable at 20° C., to their use and to a process for the production of emulsions using the emulsifier concentrate.

### PRIOR ART

The production of stable preparations in emulsion form normally involves a considerable outlay on equipment because the emulsification of the oil and water phases generally has to be carried out at elevated temperatures. The solid and wax-like components of the oil phase which have to be melted include inter alia a number of w/o emulsifiers. These solid materials are far more difficult to dose in industrial-scale processes than liquid components. The wax-like w/o emulsifiers commonly used in cosmetic products include the polyethylene glycol diesters (PEG diesters). However, many of these are insoluble at room temperature in the oil components typically used in cosmetic preparations. Even with a ratio of 10% by weight PEG diester to 90% by weight oil components, a deposit or paste-like or wax-like products is/are formed.

The problem addressed by the present invention was to provide PEG diester concentrates which would be flowable and pumpable at 20° C. and which could readily be incorporated in emulsion-form preparations. The PEG diester content of the concentrate would be very high and the concentrate would even lend itself to cold processing.

### DESCRIPTION OF THE INVENTION

The present invention relates to an emulsifier concentrate flowable at 20° C. containing
(a) up to 90% by weight of a polyethylene glycol fatty acid diester based on a C₁₆₋₂₂ fatty acid, a C₁₆₋₂₂ hydroxyfatty acid, polyhydroxystearic acid or poly-(C₁₆₋₂₂ hydroxyfatty acid),
(b) up to 80% by weight of an oil component liquid at 25° C. or a mixture of such oil components and
(c) 2 to 8% by weight water.

The concentrates preferably no contain other constituents except for impurities attributable to the raw materials used, so that they consist essentially of components (a) to (c). Component (a) is preferably present in the concentrate in a quantity of up to 85% by weight. In a preferred embodiment, the emulsifier concentrate contains (a) 10 to 80% by weight of a polyethylene glycol fatty acid diester based on a C₁₆₋₂₂ fatty acid, a C₁₆₋₂₂ hydroxyfatty acid, polyhydroxystearic acid or poly-(C₁₆₋₂₂ hydroxyfatty acid), (b) 10 to 80% by weight of an oil component liquid at 25° C. or a mixture of such oil components and (c) 2 to 8% by weight water. Particularly preferred concentrates contain (a) 30 to 60% by weight of a polyethylene glycol fatty acid diester based on a C₁₆₋₂₂ fatty acid, a C₁₆₋₂₂ hydroxyfatty acid, polyhydroxystearic acid or poly-(C₁₆₋₂₂ hydroxyfatty acid), (b) 30 to 60% by weight of an oil component liquid at 25° C. or a mixture of such oil components and (c) 3 to 6% by weight water. A most particularly preferred concentrate has the following composition: (a) 35 to 55% by weight of a polyethylene glycol fatty acid diester based on a C₁₆₋₂₂ fatty acid, a C₁₆₋₂₂ hydroxyfatty acid, polyhydroxystearic acid or poly-(C₁₆₋₂₂ hydroxyfatty acid), (b) 40 to 60% by weight of an oil component liquid at 25° C. or a mixture of such oil components and (c) 3 to 6% by weight water.

It has surprisingly been found that the polyethylene glycol fatty acid diesters (a) dissolve in liquid oil components if a defined quantity of water is added. The quantity of water is a critical parameter. Emulsifier concentrates containing quantities of up to 90% by weight of a polyethylene glycol fatty acid diester are soluble in oil components of various different polarities if 2 to 8% by weight water is present in the concentrates. With a water content below 2% by weight, a deposit is formed or the mixture assumes a paste-like to solid consistency. With a water content above 8% by weight, the mixture gels and the concentrates are no longer pumpable or flowable. Quantities of water of 3 to 6% by weight, based on the total quantity of concentrate, are preferred while quantities of water of 4 to 5% by weight are particularly preferred.

A preferred embodiment of the emulsifier concentrate is characterized in that it contains no other nonionic, anionic, cationic, amphoteric and/or zwitterionic surfactants. In particular, the concentrate according to the invention does hot contain any UV filters and/or pigments. In a preferred embodiment, the concentrate consists essentially of components (a), (b) and (c). The polyethylene glycol diester may contain residues of monoester, unesterified fatty acid, polyethylene glycol and polyethylene glycol monoester from its production. The polyethylene glycol diesters themselves have been known for some time and are available from numerous suppliers. The PEG unit of the polyethylene glycol diesters of the concentrates according to the invention has a molecular weight of 600 to 3,000, preferably in the range from 1,000 to 2,000 and more particularly of the order of ca. 1,500. This corresponds on average to 30 recurring oxyethylene units.

Particularly suitable emulsifier concentrates contain as component a) an ester obtainable by esterification of a fatty acid and/or hydroxyfatty acid containing 16 to 22 carbon atoms or a corresponding polyfatty acid and/or polyhydroxyfatty acid having a degree of self-condensation of 2 to 20 and more particularly 2 to 10 with a polyethylene glycol in the presence of a catalyst comprising an inorganic phosphorus(I) compound and a titanate, the carbonyl compound and the inorganic phosphorus(I) compound being mixed with one another, the mixture obtained being filtered and the alcohol and the titanate being added to the filtered mixture and the esterification reaction being carried out. One such process, which gives particularly pure and colorless PEG diesters, is the subject of DE 102 51 984.

According to the invention, preferred emulsifier concentrates have a viscosity at 20° C. of less than 20,000 mpa.s (Brookfield viscosimeter, spindle 5, 10 r.p.m.). Particularly preferred emulsifier concentrates have a viscosity at 15° C. of less than 10,000 mPa.s. In another preferred embodiment, the emulsifier concentrates are transparent or translucent.

Oil Components

Suitable oil components for the emulsion concentrate are any of the oils liquid at 25° C./normal pressure which are typically used in cosmetic preparations or mixtures of such oils. Examples of suitable oil components are Guerbet alcohols based on fatty alcohols containing 6 to 18 and preferably 8 to 10 carbon atoms, esters of linear C₆₋₂₂ fatty acids with linear or branched C₁₆₋₂₂ fatty alcohols or esters of branched C₆₋₁₃ carboxylic acids with linear or branched C₆₋₂₂ fatty alcohols such as, for example, hexyl laurate, myristyl isostearate, myristyl oleate, cetyl isostearate, cetyl oleate, stearyl isostearate, stearyl oleate, isostearyl myristate, isostearyl palmitate, isostearyl stearate, isostearyl isostearate, isostearyl oleate, oleyl myristate, oleyl isostearate, oleyl oleate, oleyl erucate, erucyl isostearate, erucyl oleate, cococaprylate/caprate. Also suitable are esters of linear C₆₋₂₂ fatty acids with branched alcohols, more particularly 2-ethyl hexanol and isopropanol, esters of C₁₈₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, liquid triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, such as Dicaprylyl Carbonate (Cetiol® CC) for example, Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols, mixtures of these oil components with silicone oils (cyclomethicone, silicon methicone types, etc.) and/or aliphatic or naphthenic hydrocarbons such as, for example, mineral oil, Vaseline, petrolatum, isohexadecanes, squalane, squalene or dialkyl cyclohexanes. Preferred emulsifier concentrates according to the invention contain oil components selected from the group consisting of dialkyl ethers, dialkyl carbonates, triglycerides, esters, hydrocarbons, branched C₁₂₋₂₄ fatty alcohols or a mixture of these substances. In a particularly preferred embodiment, the oil component consists essentially of dialkyl carbonates or dialkyl ethers or a mixture of these components, in other words other oil components are present as impurities solely from the production process. In another particularly preferred embodiment, the oil component is dioctyl ether or dioctyl carbonate or a mixture of these two substances.

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. Another preferred embodiment of the emulsifier concentrate is characterized in that it contains a hydrotrope selected from the group of polyols containing 2 to 15 carbon atoms and at least 2 hydroxyl groups. These hydrotropes also improve the cold flow behavior of the concentrate.

Cosmetic Preparations

The emulsifier concentrates according to the invention enable emulsions to be produced in a cold process providing all other constituents are liquid. The present invention also relates to the use of the emulsifier concentrates according to the invention in cosmetic and pharmaceutical emulsions. The invention also relates to a process for the production of emulsions which is characterized in that the emulsifier concentrate according to the invention is emulsified in a hot or cold process with an oil phase and a water phase which may contain the usual auxiliaries and additives and other emulsifiers. These emulsions are preferably body care formulations, for example in the form of creams, milks, lotions, sprayable emulsions, products for eliminating body odor, etc. The compound according to the invention may also be used in surfactant-containing formulations such as, for example, foam and shower baths, hair shampoos and care rinses.

The cosmetic preparations may be formulated as emulsions or dispersions. Preferred cosmetic compositions are those in the form of a w/o or o/w emulsion with the usual concentrations—known to the expert—of oils/fats/waxes, emulsifiers, water and the other auxiliaries and additives typically used in cosmetic preparations.

Depending on the particular application envisaged, the cosmetic formulations contain a number of other auxiliaries and additives, such as, for example, surface-active substances (surfactants, emulsifiers), other oil components, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, antidandruff agents, film formers, swelling agents, insect repellents, self-tanning agents, tyrosinase inhibitors (depigmenting agents), hydrotropes, solubilizers, preservatives, perfume oils, dyes, etc. of which some are listed by way of example in the following.

The quantities of the particular additives are governed by the particular application envisaged.

Surface-Active Substances

The surface-active substances present may be anionic, nonionic, cationic and/or amphoteric or zwitterionic surfactants or emulsifiers or a mixture thereof. In surfactant-containing cosmetic preparations such as, for example, shower gels, foam baths, shampoos, etc., at least one anionic surfactant is preferably present. Body-care creams and lotions preferably contain other nonionic surfactants/emulsifiers.

Typical examples of anionic surfactants are soaps, alkyl benzene-sulfonates, alkanesulfonates, olefin sulfonates, alkylether sulfonates, glycerol ether sulfates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants/emulsifiers are fatty alcohol polyglycol ethers, polyglycerol esters, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, amino-propionates, aminoglycinates, imidazolinium betaines and sulfobetaines. The surfactants mentioned are all known compounds. Information on their structure and production can be found in relevant synoptic works in this field. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligo-glucosides, fatty acid glucamides, alkylamidobetaines, amphoacetals and/or protein fatty acid condensates, preferably based on wheat proteins.

Oil Components

Body care preparations, such as creams, lotions, normally contain a number of other oil components and emollients which contribute towards further optimizing their sensory properties. Any oil components suitable for cosmetic applications may be incorporated in the final cosmetic formulations. Any of the oil components mentioned as examples of oil components for the emulsifier concentrate (vide supra) are suitable for this purpose.

In addition, the cosmetic compositions may also contain other hydrotropes of the type already mentioned for the concentrate.

Fats and Waxes

Fats and waxes are added to the body care products both as care components and to increase the consistency of the cosmetic preparations. Typical examples of fats are glycerides, i.e. solid or liquid, vegetable or animal products which consist essentially of mixed glycerol esters of higher fatty acids. Fatty acid partial glycerides, i.e. technical mono- and/or di-esters of glycerol with C₁₂₋₁₈ fatty acids, such as for example glycerol mono/dilaurate, palmitate or stearate, may also be used for this purpose. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Thickeners

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, polyacrylates (for example Carbopols® and Pemulen types [Goodrich]; Synthalens® [Sigma]; Keltrol types [Kelco]; Sepigel types [Seppic]; Salcare types [Allied Colloids] and Cosmedia® SP and SPL [Cognis]), polyacrylamides, polymers, polyvinyl alcohol, polyvinyl pyrrolidone and bentonites, for example Bentone® Gel VS-5PC (Rheox). Other suitable thickeners are electrolytes, such as sodium chloride and ammonium chloride.

Stabilizers

Metal salts of fatty acids such as, for example, magnesium, aluminum and/or zinc stearate or ricinoleate may be used as stabilizers.

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. Typical UV-A filters are, in particular, derivatives of benzoyl methane. The UV-A and UV-B filters may of course also be used in the form of mixtures, for example combinations of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene), and esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide. Silicates (talcum), barium sulfate and zinc stearate may be used as salts. The oxides and salts are used in the form of the pigments for skin-care and skin-protecting emulsions.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin.

Biogenic Agents

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes.

Deodorizing Agents

Deodorizing agents counteract, mask or eliminate body odors. Body odors are formed through the action of skin bacteria on apocrine perspiration which results in the formation of unpleasant-smelling degradation products. Accordingly, suitable deodorizing agents are inter alia germ inhibitors, enzyme inhibitors, odor absorbers or odor maskers.

Antiperspirants

Antiperspirants reduce perspiration and thus counteract underarm wetness and body odor by influencing the activity of the eccrine sweat glands. Suitable astringent active principles of antiperspirants are, above all, salts of aluminum, zirconium or zinc. Suitable antihydrotic agents of this type are, for example, aluminum chloride, aluminum chlorohydrate, aluminum dichlorohydrate, aluminum sesquichlorohydrate and complex compounds thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, for example with amino acids, such as glycine.

Insect Repellents

Suitable insect repellents are N,N-diethyl-m-toluamide, pentane-1,2-diol or 3-(N-n-butyl-N-acetylamino)-propionic acid ethyl ester), which is marketed under the name of Insect Repellent® 3535 by Merck KGaA, and butyl acetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Preservatives

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, and synthetic perfume compounds of the ester, ether, aldehyde, ketone, alcohol and hydrocarbon type may also be used.

Dyes

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). These dyes are normally used in concentrations of 0.001 to 0.1% by weight, based on the mixture as a whole.

### EXAMPLES

The invention is illustrated by the following Examples.
I. Synthesis of the Polyethylene Glycol Fatty Acid Diesters

### Example A

Synthesis of a polyethylene glycol-1500-polyhydroxy stearate

726.8 g (2.37 mol) 12-hydroxystearic acid, 273.2 g (0.189 mol) polyethylene glycol 1500 and 0.2 g tetrabutyl titanate are heated in stages under nitrogen to 240° C. After the separation of water has ended, a vacuum is applied and the condensation reaction is continued until there is no further reduction in the acid value. After cooling to 100° C. and addition of 0.5% filter aid (Hyflow® Supercel), the product is filtered.

The product (filtrate) has an acid value of 9.5. It is dark brown in color. Neither the Hazen nor the Gardner color value can be determined. The product has the consistency (at 20° C.) of a wax-like solid.

### Example B

Synthesis of a polyethylene glycol-1000-polyhydroxy stearate

754.3 g (2.46 mol) 12-hydroxystearic acid, 245.8 g (0.246 mol) polyethylene glycol 1000 and 0.1 g tin oxide are heated in stages under nitrogen to 240° C. After the separation of water has ended, a vacuum is applied and the condensation reaction is continued until there is no further reduction in the acid value. After cooling to 100° C. and addition of 0.5% filter aid (Hyflow® Supercel), the product is filtered.

The product (filtrate) has an acid value of 17. It is dark brown in color. Neither the Hazen nor the Gardner color value can be determined. The product has the consistency (at 20° C.) of a wax-like solid.

### Example C

Synthesis of a polyethylene glycol-3000-polyhydroxy stearate

506 g (1.65 mol) 12-hydroxystearic acid, 494 g (0.165 mol) polyethylene glycol 3000 and 0.1 g tin oxide are heated in stages under nitrogen to 240° C. After the separation of water has ended, a vacuum is applied and the condensation reaction is continued until there is no further reduction in the acid value. After cooling to 100° C. and addition of 0.5% filter aid (Hyflow® Supercel), the product is filtered.

The product (filtrate) has an acid value of 18. It is dark brown in color. Neither the Hazen nor the Gardner color value can be determined. The product has the consistency (at 20° C.) of a wax-like solid.

### Example D

Synthesis of a polyethylene glycol-1500-polyhydroxy stearate

5 g phosphorus(l) acid (50%) are added to 726.8 g (2.37 mol) 12-hydroxystearic acid, followed by stirring for 1 hour at 90° C. After addition of 8 g sodium carbonate and 5 g Hyflow® Supercel, the hot mixture is filtered. 273.2 g (0.189 mol) polyethylene glycol 1500 and 0.4 g Tyzor® TBT are added to the filtrate. The reaction mixture is slowly heated under nitrogen to 190° C. over a period of 2 hours and esterified for 18 hours with continuous removal of water in vacuo, the temperature being gradually increased to 210° C. After cooling to ca. 100° C. and filtration, the product is obtained as the filtrate.

The product has an acid value of 8, an iodine value of 2 and a Hazen color value of 100. It has the consistency (at 20° C.) of a wax-like solid.

### Example E

Synthesis of a polyethylene glycol-600-polyhydroxy stearate

836.5 g (2.72 mol) 12-hydroxystearic acid, 163.5 g (0.272 mol) polyethylene glycol 600 and 0.1 g tin oxide are heated in stages under nitrogen to 240° C. After the separation of water has ended, a vacuum is applied and the condensation reaction is continued until there is no further reduction in the acid value. After cooling to 100° C. and addition of 0.5% filter aid (Hyflow® Supercel), the product is filtered.

The product (filtrate) has an acid value of 18. It is dark brown in color. Neither the Hazen nor the Gardner color value can be determined. The product has the consistency (at 20° C.) of a wax-like solid.

### Example F

Synthesis of a polyethylene glycol-3000-polyhydroxy stearate

711 g (2.32 mol) 12-hydroxystearic acid, 289 g (0.483 mol) polyethylene glycol 3000 and 0.2 g tin tetrabutyl titanate are heated in stages under nitrogen to 240° C. After the separation of water has ended, a vacuum is applied and the condensation reaction is continued until there is no further reduction in the acid value. After cooling to 100° C. and addition of 0.5% filter aid (Hyflow® Supercel), the product is filtered.

The product (filtrate) has an acid value of 18. It is dark brown in color. Neither the Hazen nor the Gardner color value can be determined. The product has the consistency (at 20° C.) of a wax-like solid.
II. Production of the Emulsifier Concentrates According to the Invention Using the Polyethylene Glycol Fatty Acid Diesters

The liquid polyethylene glycol polyhydroxystearate mixtures were produced by the following process: the oil (or oil mixture) intended for compounding is introduced into the mixing vessel and the molten polyethylene glycol is added. The mixture is homogenized, optionally after further heating, and other non-aqueous additives are optionally added with stirring. Finally, the water is added with stirring.

### Example 1

A compound consisting of 55 g di-n-butyl carbonate and 41 g of the polyethylene glycol fatty acid diester of Example A and 4 g deionized water is prepared by the mixing process described above. The mixture is a clear liquid at 20° C. (Brookfield viscosity at 20° C.: ca. 2,000 mPa.s; spindle 5/10 r.p.m.).

### Example 2

A mixture consisting of 40.5 g di-n-octyl carbonate and 55 g of the polyethylene glycol fatty acid diester of Example A and 4.5 g deionized water is prepared by the mixing process described above. The mixture is a clear liquid at 20° C. (Brookfield viscosity at 20° C.: 9,000 mPa.s; spindle 5/10 r.p.m.).

### Example 3

A mixture consisting of 19.5 g di-n-octyl carbonate and 77.5 g of the polyethylene glycol fatty acid diester of Example A and 3 g deionized water is prepared by the mixing process described above. The mixture is a clear liquid at 20° C.

### Example 4

A mixture consisting of 40.5 g di-n-octyl ether and 55 g of the polyethylene glycol fatty acid diester of Example A and 4.5 g deionized water is prepared by the mixing process described above. The mixture is a clear liquid at 20° C. (Brookfield viscosity at 20° C.: 8,500 mpa.s; spindle 5/10 r.p.m.).

### Example 5

A mixture consisting of 24 g di-n-octyl ether and 73 g of the polyethylene glycol fatty acid diester of Example A and 3 g deionized water is prepared by the mixing process described above. The mixture is a clear liquid at 20° C.

### Example 6

A compound consisting of 55 g di-n-octyl carbonate and 41 g of the polyethylene glycol fatty acid diester of Example D and 4 g deionized water is prepared by the mixing process described above. The mixture is a clear liquid at 15° C. (Brookfield viscosity at 20° C.: ca. 1,200 mpa.s; spindle 5/10 r.p.m.).

### Example 7

A compound consisting of 38 g di-n-octyl carbonate and 57 g of the polyethylene glycol fatty acid diester of Example D and 5 g deionized water is prepared by the mixing process described above. The mixture is a clear liquid at 20° C. (Brookfield viscosity at 20° C.: ca. 9,800 mPa.s; spindle 5/10 r.p.m.).

### Example 8

A mixture consisting of 39 g di-n-octyl carbonate and 57 g of the polyethylene glycol fatty acid diester of Example D and 4 g deionized water is prepared by the mixing process described above. The mixture is a clear liquid at 20° C. (Brookfield viscosity at 20° C.: ca. 5,900 mPa.s; spindle 5/10 r.p.m.).

### Example 9

A mixture consisting of 40.5 g di-n-octyl carbonate and 55.0 g of the polyethylene glycol fatty acid diester of Example D and 4.5 g deionized water is prepared by the mixing process described above. The mixture is a clear liquid at 20° C. (Brookfield viscosity at 20° C.: ca. 6,200 mpa.s; spindle 5/10 r.p.m.).

### Example 10

A mixture consisting of 38 g di-n-octyl ether and 57 g of the polyethylene glycol fatty acid diester of Example D and 5 g deionized water is prepared by the mixing process described above. The mixture is a clear liquid at 20° C.

### Example 11

A mixture consisting of 24 g di-n-octyl ether and 73 g of the polyethylene glycol fatty acid diester of Example D and 3 g deionized water is prepared by the mixing process described above. The mixture is a clear liquid at 20° C.

### Example 12

A mixture consisting of 47.5 g Myritol 331 (Cocoglycerides) and 47.5 g of the polyethylene glycol fatty acid diester of Example D and 5 g deionized water is prepared by the mixing process described above. The mixture is a clear liquid at 20° C.

### Example 13

A mixture consisting of 55 g safflower oil and 41 g of the polyethylene glycol fatty acid diester of Example D and 4 g deionized water is prepared by the mixing process described above. The mixture is a clear liquid at 20° C. (Brookfield viscosity at 20° C.: ca. 9,000 mPa.s; spindle 5/10 r.p.m.).

### Example 14

A mixture consisting of 55 g Eutanol G (Octyl Dodecanol) and 41 g of the polyethylene glycol fatty acid diester of Example D and 4 g deionized water is prepared by the mixing process described above. The mixture is a clear liquid at 20° C. (Brookfield viscosity at 20° C.: ca. 9,000 mpa.s; spindle 5/10 r.p.m.).

### Example 15

A mixture consisting of 55 g paraffin oil, thinly liquid, and 41 g of the polyethylene glycol fatty acid diester of Example D and 4 g deionized water is prepared by the mixing process described above. The mixture is a clear liquid at 20° C.

### Example 16

A mixture consisting of 47.5 g di-n-octyl ether and 47.5 g of the polyethylene glycol fatty acid diester of Example E and 5 g deionized water is prepared by the mixing process described above. The mixture is a clear liquid at 20° C.

### Example 17

A mixture consisting of 47.5 g di-n-octyl ether and 47.5 g of the polyethylene glycol fatty acid diester of Example B and 5 g deionized water is prepared by the mixing process described above. The mixture is a clear liquid at 20° C.

### Example 18

A mixture consisting of 47.5 g di-n-octyl ether and 47.5 g of the polyethylene glycol fatty acid diester of Example C and 5 g deionized water is prepared by the mixing process described above. The mixture is a clear liquid at 20° C.

### Example 19

A mixture consisting of 47.5 g di-n-octyl ether and 47.5 g of the polyethylene glycol fatty acid diester of Example F and 5 g deionized water is prepared by the mixing process described above. The mixture is a clear liquid at 20° C.

### Example 20

A mixture consisting of 46 g di-n-octyl carbonate, 46 g of the polyethylene glycol fatty acid diester of Example C, 4 g glycerol and 4 g deionized water is prepared by the mixing process described above. The mixture is a clear liquid at 20° C. (Brookfield viscosity at 20° C.: ca. 3,000 mPa.s; spindle 5/10 r.p.m.).

### Example 21

A mixture consisting of 42 g di-n-octyl carbonate, 52 g of the polyethylene glycol fatty acid diester of Example C, 2 g butylene glycol (butane-1,3-diol) and 4 g deionized water is prepared by the mixing process described above. The mixture is a clear liquid at 20° C. (Brookfield viscosity at 20° C.: ca. 7,000 mPa.s; spindle 5/10 r.p.m.).

### Example 22

A mixture consisting of 50.5 g di-n-octyl carbonate, 41.5 g of the polyethylene glycol fatty acid diester of Example C, 4 g butylene glycol (butane-1,3-diol) and 4 g deionized water is prepared by the mixing process described above. The mixture is a clear liquid at 20° C. (Brookfield viscosity at 20° C.: ca. 4,000 mPa.s; spindle 5/10 r.p.m.).

### Example 23

A mixture consisting of 37 g di-n-octyl carbonate, 47 g of the polyethylene glycol fatty acid diester of Example C, 10 g Eutanol G (Octyl Dodecanol) and 6 g deionized water is prepared by the mixing process described above. The mixture is a clear liquid at 20° C. (Brookfield viscosity at 20° C.: ca. 7,000 mpa.s; spindle 5/10 r.p.m.). The mixture is a clear liquid at 15° C. (Brookfield viscosity at 15° C.: ca. 9,000 mpa.s; spindle 5/10 r.p.m.).

### Example 24

A mixture consisting of 42 g di-n-octyl carbonate, 52 g of polyethylene glycol fatty acid diester of Example C, 2 g butylene glycol (butane-1,3-diol) and 4 g deionized water is prepared by the mixing process described above. The mixture is a clear liquid at 20° C. (Brookfield viscosity at 20° C.: ca. 7,000 mPa.s; spindle 5/10 r.p.m.).

### Example 25

A mixture consisting of 40 g di-n-octyl carbonate, 20 g cyclomethicone (Dow Corning 245), 36 g of the polyethylene glycol fatty acid diester of Example A and 4 g deionized water is prepared by the mixing process described above. The mixture is a clear liquid at 20° C. (Brookfield viscosity at 20° C.: ca. 1,000 mPa.s; spindle 5/10 r.p.m.).

### Example 26

A mixture consisting of 11 g Eutanol G (Octyl Dodecanol), 85 g of the polyethylene glycol fatty acid diester of Example D and 4 g deionized water is prepared by the mixing process described above. The mixture is a clear liquid at 20° C. (Brookfield viscosity at 20° C.: ca. 17,000 mPa.s; spindle 5/10 r.p.m.).

### Example 27

A mixture consisting of 8 g Eutanol G 16 (Hexyl Decanol), 88 g of the polyethylene glycol fatty acid diester of Example A and 4 g deionized water is prepared by the mixing process described above. The mixture is a clear liquid at 20° C.
III. Comparison Examples

### Comparison Example 1

A mixture consisting of 45 g di-n-octyl carbonate and 45 g of the polyethylene glycol fatty acid diester of Example A and 10 g deionized water is prepared by the mixing process described above. The mixture is a gel at 20° C. (Brookfield viscosity at 20° C.: not measurable).

### Comparison Example 2

A mixture consisting of 50 g di-n-octyl carbonate and 50 g of the polyethylene glycol fatty acid diester of Example A is prepared by the mixing process described above (without addition of water). The mixture is a wax-like solid at 20° C. (Brookfield viscosity at 20° C.: not measurable).

### Comparison Example 3

A mixture consisting of 35 g di-n-octyl ether and 55 g of the polyethylene glycol fatty acid diester of Example A and 10 g deionized water is prepared by the mixing process described above. The mixture is a gel at 20° C. (Brookfield viscosity at 20° C.: not measurable).

### Comparison Example 4

A mixture consisting of 50 g di-n-octyl ether and 50 g of the polyethylene glycol fatty acid diester of Example A is prepared by the mixing process described above (without addition of water). The mixture is a wax-like solid at 20° C. (Brookfield viscosity at 20° C.: not measurable).

### Comparison Example 5

A mixture consisting of 46 g di-n-octyl carbonate, 46 g of the polyethylene glycol fatty acid diester of Example C and 4 g glycerol is prepared by the mixing process described above (without addition of water). The mixture is a wax-like solid at 20° C. (Brookfield viscosity at 20° C.: not measurable).

### Comparison Example 6

A mixture consisting of 42 g di-n-octyl carbonate, 52 g of the polyethylene glycol fatty acid diester of Example C and 4 g butylene glycol (butane-1,3-diol) is prepared by the mixing process described above without addition of water. The mixture is a wax-like solid at 20° C. (Brookfield viscosity at 20° C.: not measurable).

### Comparison Example 7

A mixture consisting of 40 g di-n-octyl carbonate, 10 g Eutanol G (Octyl Dodecanol) and 50 g of the polyethylene glycol fatty acid diester of Example C is prepared by the mixing process described above (without addition of water). The mixture is a wax-like solid at 20° C. (Brookfield viscosity at 20° C.: not measurable).

### FORMULATION EXAMPLES

Cosmetic compositions formulated with the emulsifier concentrates are disclosed in Tables 1 and 2. The quantities of the individual components are expressed in % by weight of the commercially available substances, based on the composition as a whole.

Using selected emulsifier concentrates, various cosmetic compositions were prepared by the usual, known hot (“H”) or cold (“C”) methods.

The viscosity measurements were carried out with a Brookfield RVF viscosimeter at 23° C. (spindle 5, 10 r.p.m., 23° C. or spindle TE, 4 r.p.m., Helipath, depending on the viscosity).

The structure and stability of the formulations were evaluated by a trained test person. The criteria of “structure” and “stability” were evaluated on a scale of −2 to +2: structure (−2: very inhomogeneous to +2: very homogeneous) and stability (−2: poor to +2: high).

TABLE 1
Cosmetic emulsions

| Constituents/ | Example 1 | Example 2 | Example 3 | Example 4 | Example 5 | Example 6 | Example 7 |
|---|---|---|---|---|---|---|---|
| (INCI) | H | H | H | C | H | C | H |
| Emulsifier concen- | | | | | | | |
| trate of Example 2 | | | | | | | |
| Emulsifier concen- | | | | | | | |
| trate of Example 6 | | | | | | | |
| Emulsifier concen- | 8.33 | 3.33 | 3.33 | 3.33 | 3.33 | 3.33 | 5.0 |
| trate of Example 9 | | | | | | | |
| Lameform ® TGI | | 2.0 | | | | | |
| (Polyglyceryl-3 | | | | | | | |
| Diisostearate) | | | | | | | |
| Dehymuls ® PGPH | | | 2.0 | 2.0 | 2.0 | 2.0 | |
| (Polyglyceryl-2 | | | | | | | |
| Dipolyhydroxystearate) | | | | | | | |
| Cetiol ® CC | | 6.67 | 4.67 | 4.67 | 3.17 | 3.17 | 4.0 |
| (Dicaprylyl Carbonate) | | | | | | | |
| Cetiol ® OE | 2.67 | | | | 4.5 | 4.5 | |
| (Dicaprylyl Ether) | | | | | | | |
| Cetiol ® A | 6.0 | | 6.0 | 6.0 | | | 6.0 |
| (Hexyl Laurate) | | | | | | | |
| Cetiol ® SN | 10.0 | | 7.0 | 7.0 | 4.8 | 4.8 | 10.0 |
| (Cetearyl Isononanoate) | | | | | | | |
| Eutanol ® G16 | 3.0 | | 3.0 | 3.0 | 2.2 | 2.2 | 3.0 |
| (Hexyldecanol) | | | | | | | |
| Myritol ® 331 | | | | | | | |
| (Cocoglycerides) | | | | | | | |
| Cetiol ® OE | | | | | | | |
| (Dicaprylyl Ether) | | | | | | | |
| Cetiol ® PGL | | | | | | | |
| (Hexyldecanol and | | | | | | | |
| Hexyldecyl Laurate) | | | | | | | |
| Cetiol ® 868 | | | | | | | |
| (Octyl Stearate) | | | | | | | |
| Isopropylmyristate | | 8.0 | | | | | |
| Vaseline | | 2.0 | | | | | |
| Zincum ® N29 | | 1.0 | | | | | |
| (Zinc Stearate) | | | | | | | |
| Rezal ® 36GP | | | | | 40.0 | 40.0 | |
| (Aluminum Zirconium | | | | | | | |
| Tetrachlorohydrex GLY) | | | | | | | |
| Copherol ® F1300 | | | | | | | |
| (Tocopherol) | | | | | | | |
| 1,3-Butylene glycol | | 3.0 | | | | | |
| MgSO₄•7H₂O | 1.0 | 1.0 | 1.0 | 1.0 | 1.0 | 1.0 | 1.0 |
| Magnesium Sulfate | | | | | | | |
| Glycerol, 99.5% | 3.0 | 2.0 | 3.0 | 3.0 | 3.0 | 3.0 | 3.0 |
| Water, deionized | to 100 | to 100 | to 100 | to 100 | to 100 | to 100 | to 100 |
| Preservative | | | | | | | |
| Evaluation | | | | | | | |
| Structure of the | +2 | +2 | +2 | +2 | +2 | +2 | +2 |
| emulsion | | | | | | | |
| Viscosity of the | 1200 | 13600 | 5200 | 11200 | 6800 | 20000 | 1600 |
| emulsion | | | | | | | |
| Stabilities at RT/−5° C./ | +2 | +2 | +2 | +2 | +2 | +2 | +2 |
| 40° C./45° C./50° C. | | | | | | | |
| (over 12 weeks) | | | | | | | |

The stabilities were given a score of +2 at all four temperatures.
H: production by hot process
C: production by cold process

TABLE 2

| Constituents/ | Ex. 8 | Ex. 9 | Ex. 10 | Ex. 11 | Ex. 12 | Ex.13 | Ex. 14 | Ex. 15 | Ex. 16 |
|---|---|---|---|---|---|---|---|---|---|
| (INCI) | H | H | H | C | H | C | H | H | H |
| Emulsifier con- | | | 3.33 | 3.33 | 3.33 | 3.33 | | | |
| centrate of Ex. 2 | | | | | | | | | |
| Emulsifier con- | | | | | | | 7.2 | 7.2 | 7.2 |
| centrate of Ex. 6 | | | | | | | | | |
| Emulsifier con- | 7.5 | 3.5 | | | | | | | |
| centrate of Ex. 9 | | | | | | | | | |
| Lameform ® TGI | | 2.0 | | | | | | | |
| (Polyglyceryl-3 | | | | | | | | | |
| Diisostearate) | | | | | | | | | |
| Dehymuls ® PGPH | | | 2.0 | 2.0 | 2.0 | 2.0 | | | |
| (Polyglyceryl-2 Di- | | | | | | | | | |
| polyhydroxystearate) | | | | | | | | | |
| Cetiol ® CC | 2.0 | 6.5 | 4.67 | 4.67 | 3.17 | 3.17 | 1.8 | | 1.8 |
| (Dicaprylyl Carbonate) | | | | | | | | | |
| Cetiol ® OE | | | | | 4.5 | 4.5 | | 1.8 | |
| (Dicaprylyl Ether) | | | | | | | | | |
| Cetiol ® A | | | 6.0 | 6.0 | | | 6.0 | 6.0 | 6.0 |
| (Hexyl Laurate) | | | | | | | | | |
| Cetiol ® SN | | | 7.0 | 7.0 | 4.8 | 4.8 | 10.0 | | |
| (Cetearyl Isononanoate) | | | | | | | | | |
| Eutanol ® G16 | | | 3.0 | 3.0 | 2.2 | 2.2 | 3.0 | 3.0 | 3.0 |
| (Hexyldecanol) | | | | | | | | | |
| Myritol ® 331 | 6.0 | | | | | | | | |
| (Cocoglycerides) | | | | | | | | | |
| Cetiol ® OE | | | | | | | | | |
| (Dicaprylyl Ether) | | | | | | | | | |
| Cetiol ® PGL | 5.0 | | | | | | | | |
| (Hexyldecanol and | | | | | | | | | |
| Hexyldecyl Laurate) | | | | | | | | | |
| Cetiol ® 868 | | | | | | | | 10.0 | 10.0 |
| (Octyl Stearate) | | | | | | | | | |
| Isopropylmyristate | | 8.0 | | | | | | | |
| Vaseline | | 2.0 | | | | | | | |
| Zincum ® N29 | 1.0 | 1.0 | | | | | | | |
| (Zinc Stearate) | | | | | | | | | |
| Rezal ® 36GP | | | | | 40.0 | 40.0 | | | |
| (Aluminum Zirconium | | | | | | | | | |
| Tetrachlorohydrex GLY) | | | | | | | | | |
| Copherol ® F1300 | 1.0 | 0.0 | | | | | | | |
| (Tocopherol) | | | | | | | | | |
| 1,3-Butylene glycol | | 3.0 | | | | | | | |
| MgSO₄•7H₂O | 0.5 | 1.0 | 1.0 | 1.0 | 1.0 | 1.0 | 1.0 | 1.0 | 1.0 |
| Magnesium Sulfate | | | | | | | | | |
| Glycerol, 99.5% | 5.0 | 2.0 | 5.0 | 5.0 | 5.0 | 5.0 | 5.0 | 5.0 | 5.0 |
| Water, deion. | to 100 | to 100 | to 100 | to 100 | to 100 | to 100 | to 100 | to 100 | to 100 |
| Preservative | | | | | | | | | |
| Evaluation | | | | | | | | | |
| Structure of the | +2 | +2 | +2 | +2 | +2 | +2 | +2 | +2 | +2 |
| emulsion | | | | | | | | | |
| Viscosity of the | *125000 | 16800 | 9600 | 7600 | 8800 | 24800 | 1600 | 1200 | 1200 |
| emulsion | | | | | | | | | |
| Stabilities at | +2 | +2 | +2 | +2 | +2 | +2 | +2 | +2 | +2 |
| RT/−5° C./40° C./ | | | | | | | | | |
| 45° C./50° C. | | | | | | | | | |
| (over 12 weeks) | | | | | | | | | |

*Brookfield RVF, TE spindle, 4 r.p.m., +23° C.
The stabilities were given a score of +2 at all four temperatures.
H: production by hot process
C: production by cold process

### Appendix

1) Hyflow® Supercel
   INCl: Kieselgur
   Manufacturer: Manville Corporation
2) Tyzor® TPT
   INCl: Tetraisopropyltitanate
   Manufacturer: E.I. du Pont de Nemours and Company

1-10. (canceled)

11. An emulsifier concentrate flowable at 20° C. containing:
(a) 10 to 90% by weight of a polyethylene glycol fatty acid diester of at least one fatty acid selected from the group consisting of C₁₆₋₂₂ fatty acids, C₁₆₋₂₂ hydroxyfatty acids, polyhydroxystearic acids and poly-(C₁₆₋₂₂ hydroxyfatty acid);
(b) 10 to 80% by weight of an oil component liquid at 25° C. or a mixture of such oil components; and
(c) 2 to 8% by weight water.

12. The emulsifier concentrate as claimed in claim 11, which does not contain any additional nonionic, anionic, cationic, amphoteric and/or zwitterionic surfactants.

13. The emulsifier concentrate as claimed in claim 11, wherein, the oil component comprises at least one member selected from the group consisting of dialkyl ethers, dialkyl carbonates, triglycerides, esters, hydrocarbons and branched C₁₂₋₂₄ fatty alcohols.

14. The emulsifier concentrate as claimed in claim 11, having a viscosity at 20° C. of less than 20,000 mPa.s, as measured with a Brookfield viscosimeter, spindle 5, at 5 to 10 r.p.m.

15. The emulsifier concentrate as claimed in claim 11, which is transparent.

16. The emulsifier concentrate as claimed in claim 11, wherein, the concentrate further comprises at least one hydrotrope selected from the group consisting of polyols containing 2 to 15 carbon atoms and at least 2 hydroxyl groups.

17. The emulsifier concentrate as claimed in claim 11, wherein, component a) comprises an ester obtained by a process of esterifying at least one carbonyl compound selected from the group consisting of fatty acids containing 16-22 carbon atoms, hydroxyfatty acids containing 16 to 22 carbon atoms, a polyfatty acid and a poly-(C₁₆₋₂₂-hydroxyfatty acid) having a degree of self-condensation of 2 to 20 with polyethylene glycol in the presence of a catalyst comprising an inorganic phosphorus(I) compound and a titanate, which process comprises:
a) mixing the carbonyl compound with the inorganic phosphorus(I) compound to form a mixture;
b) filtering the mixture to form a filtered mixture; and
c) adding an alcohol and the titanate to the filtered mixture; and
d) carrying out the esterification reaction.

18. The process for the production of emulsions, which comprises emulsifying an oil phase and a water phase with the emulsifier concentrate claimed in claim 11 by a hot or cold process.

19. A cosmetic or pharmaceutical emulsion comprising the emulsifier concentrate of claim 11.

20. The cosmetic preparation of claim 19 containing 1 to 30% by weight of the emulsifier concentrate.

21. The emulsifier concentrate of claim 12, wherein, the oil component comprises at least one member selected from the group consisting of dialkyl ethers, dialkyl carbonates, triglycerides, esters, hydrocarbons and branched C₁₂₋₂₄ fatty alcohols.

22. The emulsifier concentrate of claim 12, having a viscosity at 20° C. of less than 20,000 mPa.s, as measured with a Brookfield viscosimeter, spindle 5, at 5 to 10 r.p.m.

23. The emulsifier concentrate of claim 12, which is transparent.

24. The emulsifier concentrate of claim 12, wherein, the concentrate further comprises at least one hydrotrope selected from the group consisting of polyols containing 2 to 15 carbon atoms and at least 2 hydroxyl groups.

25. The emulsifier concentrate of claim 12, wherein, component a) comprises an ester obtained by a process of esterifying at least one carbonyl compound selected from the group consisting of fatty acids containing 16-22 carbon atoms, hydroxyfatty acids containing 16 to 22 carbon atoms, a polyfatty acid and a poly-(C₁₆₋₂₂-hydroxyfatty acid) having a degree of self-condensation of 2 to 20 with polyethylene glycol in the presence of a catalyst comprising an inorganic phosphorus(I) compound and a titanate, which process comprises:
a) mixing the carbonyl compound with the inorganic phosphorus(I) compound to form a mixture;
b) filtering the mixture to form a filtered mixture; and
c) adding an alcohol and the titanate to the filtered mixture; and
d) carrying out the esterification reaction.

26. The process for production of emulsion, which comprises: emulsifying an oil phase and a water phase with the emulsifier concentrate claimed in claim 12.

27. The cosmetic or pharmaceutical emulsion comprising the emulsifier of claim 12.

28. The cosmetic preparation of claim 27 containing 1 to 30% by weight of the emulsifier concentrate.

29. The emulsifier concentrate as claimed in claim 13 having a viscosity at 20° C. of less than 20,000 mpa.s, as measured with a Brookfield viscosimeter, spindle 5, at 5 to 10 r.p.m.

30. The emulsifier concentrate of claim 13 which is transparent.
